(19) United States
(12) Patent Application Publication
(10) Pub. No.: US 2022/0326198 A1
(43) Pub. Date: Oct. 13, 2022

(54) QUANTIFYING PEANUT PROTEINS IN FOOD PRODUCTS

(71) Applicant: MARS, INCORPORATED, McLean, VA (US)

(72) Inventors: Yuwei CHANG, Beijing (CN); Guangtao ZHANG, Beijing (CN); Hong PENG, Beijing (CN)

(21) Appl. No.: 17/640,576

(22) PCT Filed: Sep. 12, 2019
(86) PCT No.: PCT/CN2019/105772

§ 371 Date: Mar. 4, 2022

(51) Int. Cl. G01N 30/72; G01N 1/34; G01N 30/06; G01N 33/02

(52) U.S. Cl. CPC G01N 30/7233; G01N 1/34; G01N 30/06; G01N 33/02; G01N 2030/8831

(57) ABSTRACT

Provided are methods for quantifying peanut proteins in food products using a matrix-specific calibration curve. Methods provided include exposing a food sample comprising a matrix and one or more peanut proteins to an extractant to extract the one or more peanut proteins from the food sample; heating the food sample to reduce protein interference in the food sample; digesting one or more peanut proteins in the food sample to form one or more peanut peptides; determining a relative response value of the one or more peanut peptides in the food sample by analyzing the food sample using liquid chromatography with tandem mass spectrometry; and determining an amount of the one or more peanut proteins by comparing the determined relative response value of the one or more peanut peptides in the food sample to a matrix-specific calibration curve.

### FIELD OF THE INVENTION

This disclosure relates to methods of quantifying peanut proteins in food products, and more particularly, to methods of quantifying peanut proteins in food products using a matrix-specific calibration curve that is built from relative response values of peanut peptides over a range of peanut protein concentrations for the specific matrix based on liquid chromatography with tandem mass spectrometry (LC-MSMS).

### BACKGROUND OF THE INVENTION

A peanut allergy is considered one of the most severe types of food allergies due to its prevalence, persistency, and potential severity of a reaction. Symptoms of peanut allergies can include itchiness, hives, swelling, sneezing, asthma, abdominal pain, cardiac arrest, and anaphylaxis. Thus, it is important for people who suffer from peanut allergies to know whether the food they choose to eat contains peanuts. For some, even trace amounts of peanuts can trigger a reaction.

Several different peanut proteins have been identified as potentially causing a reaction in peanut allergy sufferers. Specifically, the various proteins have been categorized into four common food allergy superfamilies: Cupin (Ara h 1, 3), Prolamin (Ara h 2, 6, 7, 9), Profilim (Ara h 5), and Bet v-1-related proteins (Ara h 8). Of these proteins, the specific proteins Ara h 1, Ara h 2, Ara h 3, and Ara h 6 trigger a reaction in more than fifty percent of the allergic population.

Currently, enzyme-linked immunosorbent assay (ELISA) is the most common method of quantifying peanut proteins in food.

### SUMMARY OF THE INVENTION

Provided are methods of quantifying peanut proteins in food products comprising a matrix and peanut proteins. In particular, methods of quantifying peanut proteins in food products provided herein can adjust the interference effects of the matrix, achieving a more sensitive and accurate method for quantifying peanut proteins in food products than conventional methods. Methods provided herein can utilize high performance liquid chromatography and tandem mass spectrometry (LC-MSMS) to measure relative response values of peanut peptides in a food product. These LC-MSMS relative response values can then be compared to a calibration curve specific to the matrix to determine the amount of peanut proteins in the food product.

Methods of quantifying peanut proteins in food products provided herein can detect and quantify peanut proteins more accurately than current ELISA methods. Using ELISA to quantify peanut proteins in food products can lead to underestimation in thermally-processed food products and/or overestimation in complex food products. Accordingly, methods for quantifying peanut proteins in food products provided herein may be more accurate and/or sensitive than ELISA methods. Because peanut proteins can elicit a severe reaction in some people, more accurate and sensitive methods of quantifying peanut proteins in food products are desirable.

In some embodiments, methods provided herein can detect and quantify peanut proteins in raw food products. In some embodiments, methods provided herein can detect and quantify peanut proteins in cooked food products. In some embodiments, methods for quantifying peanut proteins provided herein include generating a calibration curve specific to a particular matrix. For example, a calibration curve specific to a particular matrix can include relative response values of peanut peptides determined by LC-MSMS over a range of peanut protein concentrations for the specific matrix. In some embodiments, methods provided herein may not include reduction and/or alkylation of the peanut proteins.

In some embodiments, methods of quantifying peanut proteins provided herein include identifying and quantifying the peptide markers DLAFPGSGEQVEK (Ara h 1), IFLAGDKDNVIDQIEK (Ara h 1), and/or TANDLNLLILR (Ara h 3). Matrix-specific calibration curves may be generated by using the relative peak areas of these peanut peptides against concentrations of Ara h 1 and/or Ara h 3.

Accordingly, provided herein are methods for quantifying peanut proteins in food products that can more accurately identify and quantify the presence of peanut proteins in food products comprising a matrix and one or more peanut proteins. Methods provided herein can also offer a simplified approach to quantify peanut proteins by using a protein calibration curve specific to a particular matrix.

In some embodiments, a method of quantifying peanut proteins in a food sample is provided, the method comprising: exposing a food sample comprising a matrix and one or more peanut proteins to an extractant to extract the one or more peanut proteins from the food sample; heating the food sample to reduce protein interference in the food sample; digesting one or more peanut proteins in the food sample to form one or more peanut peptides; determining a relative response value of the one or more peanut peptides in the food sample by analyzing the food sample using liquid chromatography with tandem mass spectrometry (LC-MSMS); and determining an amount of the one or more peanut proteins in the food sample by comparing the determined relative response value of the one or more peanut peptides in the food sample to a matrix-specific calibration curve.

In some embodiments of the method, the matrix of the food sample comprises wheat flour.

In some embodiments of the method, the matrix of the food sample comprises one or more interference proteins.

In some embodiments of the method, exposing a food sample comprising a matrix and one or more peanut proteins to an extractant comprises extracting the one or more peanut proteins and one or more interference proteins from the food sample.

In some embodiments of the method, the matrix-specific calibration curve is specific to a matrix comprising wheat flour.

In some embodiments of the method, the matrix comprises one or more raw food ingredients.

In some embodiments of the method, the matrix comprises one or more cooked food ingredients.

In some embodiments of the method, the one or more peanut proteins comprises Ara h 1.

In some embodiments of the method, the one or more peanut proteins comprises Ara h 3.

In some embodiments of the method, heating the food sample to reduce protein interference in the food sample comprises heating the food sample to a temperature that is determined based on the matrix of the food sample.

In some embodiments of the method, the matrix-specific calibration curve comprises LC-MSMS relative response values of the one or more peanut peptides in the matrix over a range of a peanut protein concentrations.

In some embodiments of the method, the extractant comprises a tris buffer having a pH of 7.5-8.5.

In some embodiments, a method of quantifying peanut proteins in a food sample is provided, the method comprising: creating a calibration curve specific to a matrix and comprising liquid chromatography with tandem mass spectrometry (LC-MSMS) relative response values of one or more peanut peptides in the matrix over a range of one or more peanut protein concentrations; and determining, from a food sample comprising a matrix and one or more peanut proteins, an amount of the one or more peanut proteins by comparing a relative response value of one or more peanut peptides in the food sample determined by LC-MSMS to the calibration curve.

In some embodiments of the method, the matrix of the food sample comprises wheat flour.

In some embodiments of the method, the matrix of the food sample comprises one or more interference proteins.

In some embodiments of the method, the calibration curve comprises LC-MSMS relative response values of one or more peanut peptides in a matrix comprising wheat flour for a range of peanut protein concentrations.

In some embodiments of the method, the matrix comprises one or more raw food ingredients.

In some embodiments of the method, the matrix comprises one or more cooked food ingredients.

In some embodiments of the method, the one or more peanut proteins comprises Ara h 1.

In some embodiments of the method, the one or more peanut proteins comprises Ara h 3.

In some embodiments of the method, heating the food sample to reduce protein interference in the food sample.

In some embodiments of the method, heating the food sample comprises heating the food sample to a temperature that is determined based on the matrix of the food sample.

In some embodiments of the method, exposing the food sample to an extractant to extract the one or more peanut proteins from the food sample.

In some embodiments of the method, the extractant comprises a tris buffer having a pH of 7.5-8.5.

In some embodiments of the method, digesting the one or more peanut proteins in the food sample to form one or more peanut peptides.

In some embodiments of the method, determining a relative response value of the one or more peanut peptides in the food sample by analyzing the food sample using LC-MSMS.

### BRIEF DESCRIPTION OF THE DRAWINGS

Various embodiments are described with reference to the accompanying figures, in which:

FIG. 1 provides a process diagram of a method for quantifying peanut proteins in a food product, according to some embodiments;

FIG. 2 provides a process diagram of a method for quantifying peanut proteins in a food product, including developing a calibration curve, according to some embodiments;

FIG. 3 is a chart providing mass spectrometry response results showing the effects of heating on the detectability of specific peanut peptides;

FIG. 4 is a chart providing mass spectrometry response results showing the effects of heating time on the detectability of specific peanut peptides;

FIG. 5 is a chart providing mass spectrometry response results showing the effects of nitrogen blowing on the detectability of specific peanut peptides;

FIG. 6 is a chart providing mass spectrometry response results showing the effects of different extraction pH values on the detectability of specific peanut peptides;

FIGS. 7A and 7B are charts providing mass spectrometry response results showing the effects of the amount of trypsin on the detectability of specific peanut peptides; and

FIG. 8 is a chart providing mass spectrometry response results showing the effects of the amount of acetonitrile in the reconstitution solvent on the detectability of specific peanut peptides.

### DETAILED DESCRIPTION OF THE INVENTION

Described herein are methods of quantifying peanut proteins in food products comprising a matrix and one or more peanut proteins and methods of generating calibration curves specific to a particular matrix. In particular, methods of quantifying peanut proteins provided herein can utilize a calibration curve specific to a particular matrix, resulting in a more sensitive and accurate method of quantifying peanut proteins in a food product than conventional methods of quantifying peanut proteins. Methods can also include generating a matrix-specific calibration curve having relative response values of peanut peptides over a range of peanut protein concentrations for the specific matrix determined by liquid chromatography with tandem mass spectrometry (LC-MSMS). Because calibration curves are specific to particular matrices, they are able to adjust the “noise” created by the proteins of a food product. The matrix “noise” can interfere with quantification of peanut proteins in a food product, making it more difficult to detect the peanut proteins and accurately quantify them. By minimizing this matrix “noise,” methods provided herein are able to more accurately quantify the amount of peanut proteins in a given food product.

As used herein, a “food product” or “food sample” is a composition that includes both a matrix and one or more peanut proteins being measured. A “matrix” is the composition of the food product including all ingredients of the food product excluding any peanut proteins being measured. The matrix includes one or more ingredients other than the peanut proteins being measured.

In a food product comprising a matrix and one or more peanut proteins, the concentration of peanut proteins can be very low (e.g., 1 ppm or less). Thus, the concentration of non-peanut matrix proteins in the food sample (e.g., non-peanut powder proteins) can be 1 million times greater than the concentration of the peanut proteins. Due to the great discrepancy in concentrations between the peanut proteins and the non-peanut proteins of the matrix in the food product sample, the non-peanut matrix proteins interfere with the ability of quantification methods to accurately identify and quantify the trace amounts of peanut proteins.

However, methods provided herein may be more accurate and sensitive than conventional quantification methods (e.g., ELISA-based quantification methods) because the impact of these interference proteins are reduced. In particular, by measuring the relative response values of peanut peptides in a food product by LC-MSMS and comparing the relative response values of the peanut peptides to a calibration curve specific to that particular matrix to determine the amount of peanut proteins in the food product, methods provided herein are able to achieve more accurate results. In some embodiments, because a calibration curve is specific to a particular food product, it adjusts the “noise” generated by the non-peanut matrix proteins in the food product, allowing for a more accurate peanut protein quantification.

Additionally, certain techniques for preparing a food sample for analysis may help reduce the “noise” of the non-peanut matrix proteins and/or help increase the response of peanut proteins/peptides by enrichment. For example, the impact of non-peanut matrix proteins may be reduced by heating the food product and by optimizing the amount of trypsin used to digest the proteins. The extractability of peanut proteins may be increased by optimizing the pH of the extraction buffer (i.e. extractant). The peanut peptides may be concentrated by using nitrogen blowing to condense the food product, and the solubility of peanut peptides could be increased by optimizing the composition of the reconstitution solvent, and thus increasing the detectability. In each of the above examples, the detectability of one or more peanut peptides may be increased, yielding a more accurate peanut protein quantification in the food product.

Provided below is a discussion of (1) various food products and preparing food samples to be quantified, (2) extracting and digesting proteins from food samples, and (3) generating a matrix-specific calibration curve to determine the amount of peanut proteins in the food sample.

### FOOD PRODUCTS & PREPARING FOOD SAMPLES FOR QUANTIFICATION

Provided below is a discussion of food products. In particular, the food products that are discussed below may be used with methods of quantifying peanut proteins provided herein.

Food products for use with methods for quantifying peanut proteins provided herein may be any of a variety of food products that may comprise peanut proteins. For example, suitable food products include items such as cookies, bars, cakes, candy, bread, etc. Note that the food products suitable for quantification methods provided herein may be known to include peanut proteins (e.g., granola bar, peanut butter cookie, etc.). Additionally, it may be unknown whether food products suitable for quantification methods provided herein comprise peanut proteins. For example, some food products may unknowingly and/or unintentionally become contaminated with peanut proteins during manufacture or storage. Thus, a food product without an ingredient comprising peanut proteins (e.g., sugar cookie, pretzel snacks) may be analyzed by quantification methods provided herein to determine whether the food product may have inadvertently obtained trace amounts of peanut proteins.

Food products that may be analyzed using the quantification methods provided herein may comprise a matrix in addition to any peanut proteins that are present in the food product. As explained above a “food product” or “food sample” is a composition that includes both a matrix and one or more peanut proteins to be measured. A “matrix” is the composition of the food product including all ingredients of the food product excluding any peanut proteins being measured. The matrix includes one or more ingredients other than the peanut proteins being measured.

The matrix of the food product may comprise non-peanut powder, sugar (e.g., granulated, coconut, brown, confectioner's), egg, nuts (e.g., hazelnut, almond, macadamia, cashew), extracts (e.g., vanilla, almond, peppermint), milk (e.g., cow's, goat's, non-dairy), fruits and/or vegetables (e.g., apple, carrot, banana), oil, baking powder, baking soda, cocoa powder, spices (e.g., pepper, cinnamon, nutmeg, anise) and any other suitable ingredients and/or combinations thereof.

Food products for use with methods of quantifying peanut proteins provided herein may also include peanuts. For example, food products may comprise peanuts in the form of peanut powder. Peanut powder may be obtained by grinding peanuts (e.g., using a pulverizer or mortar) to a powder. In food products comprising higher amounts of peanut, the amount of peanut proteins in the food product is also greater. In food products comprising lower amounts of peanut, the amount of peanut proteins in the food product is less. In some embodiments, food products for use with methods provided herein may include between 0 and 100,000 ppm peanut. In some embodiments, a food product may include less than 100,000 ppm, less than 50,000 ppm, less than 10,000 ppm, less than 8,000 ppm, less than 6,000 ppm, less than 4,000 ppm, less than 2,000 ppm, less than 1,000 ppm, less than 800 ppm, less than 600 ppm, less than 400 ppm, less than 200 ppm, less than 100 ppm, less than 80 ppm, less than 60 ppm, less than 40 ppm, or less than 20 ppm peanut. In some embodiments, a food product may include more than 0 ppm, more than 20 ppm, more than 40 ppm, more than 60 ppm, more than 80 ppm, more than 100 ppm, more than 200 ppm, more than 400 ppm, more than 600 ppm, more than 800 ppm, more than 1,000 ppm, more than 2,000 ppm, more than 4,000 ppm, more than 6,000 ppm, more than 8,000 ppm, more than 10,000 ppm, or more than 50,000 ppm peanut. In some embodiments, the type of peanut powder used in a food product may be pure peanut powder, 80,000 ppm peanut powder, 20,000 ppm peanut powder, 4,000 ppm peanut powder, 400 ppm peanut powder, or 40 ppm peanut powder.

As described above, food products for use with methods provided herein may comprise non-peanut flour. For example, a food product may include wheat flour, whole wheat flour, oat flour, rice flour, coconut flour, almond flour, buckwheat flour, barley flour, potato flour, corn flour, amaranth flour, teff flour, arrowroot flour, and other suitable types of flour and/or combinations thereof. As the amount of non-peanut flour in a food sample increases, so too does the amount of non-peanut proteins in the food product that can interfere with methods for quantifying peanut proteins. Conversely, as the amount of non-peanut flour in the food sample decreases, so too does the amount of interference non-peanut proteins. In some embodiments, a food sample may include from 40 to 100 wt. % non-peanut flour. In some embodiments, the food sample may include less than 100 wt. %, less than 95 wt. %, less than 90 wt. %, less than 85 wt. %, less than 80 wt. %, less than 75 wt. %, less than 70 wt. %, less than 65 wt. %, less than 60 wt. %, less than 55 wt. %, less than 50 wt. %, or less than 45 wt. % non-peanut flour. In some embodiments, a food sample may include more than 40 wt. %, more than 45 wt. %, more than 50 wt. %, more than 55 wt. %, more than 60 wt. %, more than 65 wt. %, more than 70 wt. %, more than 75 wt. %, more than 80 wt. %, more than 85 wt. %, more than 90 wt. %, or more than 95 wt. % non-peanut flour.

In some embodiments, a food product may include oil. For example, suitable oils can include hydrogenated soybean oil, olive oil, vegetable oil, canola oil, coconut oil, avocado oil, sesame oil, sunflower seed oil, safflower oil, and other suitable edible oils. In some embodiments, a food sample may include between 5 and 40 wt. % oil. In some embodiments, a food sample may include less than 40 wt. %, less than 35 wt. %, less than 30 wt. %, less than 25 wt. %, less than 20 wt. %, less than 15 wt. %, or less than 10 wt. % oil. In some embodiments, a food sample may include more than 5 wt. %, more than 10 wt. %, more than 15 wt. %, more than 20 wt. %, more than 25 wt. %, more than 30 wt. %, more than 35 wt. %, more than 40 wt. %, or more than 45 wt. % oil.

In some embodiments, raw food samples may be prepared for quantification methods provided herein. The raw food samples may be prepared by measuring the individual ingredients and mixing in an appropriate mixing device. For example, the ingredients may be mixed using a grinder or pulverizer. In some embodiments, the ingredients can be mixed in intervals until a homogenous sample is obtained.

In addition to the raw food samples above, methods of quantifying peanut proteins provided herein may also be compatible with cooked food samples. For example, cooked food samples may be prepared using any of the compositions provided in Tables 3-5, provided in the Examples section below. Food samples may be cooked by roasting in an oven. In some embodiments, food samples may be cooked in an oven from 150° C. to 220° C. In some embodiments, food samples may be cooked at a temperature of less than 220° C., less than 210° C., less than 200° C., less than 190° C., less than 180° C., less than 170° C., or less than 160° C. In some embodiments, food samples may be cooked at a temperature of more than 150° C., more than 160° C., more than 170° C., more than 180° C., more than 190° C., more than 200° C., or more than 210° C.

In some embodiments, food samples may be cooked in an oven for an amount of time from 10 to 60 minutes, from 10 to 30 minutes, or from 10 to 20 minutes. In some embodiments, food samples may be cooked for less than 60 minutes, less than 50 minutes, less than 40 minutes, less than 30 minutes, or less than 20 minutes. In some embodiments, food samples may be cooked for more than 10 minutes, more than 20 minutes, more than 30 minutes, more than 40 minutes, or more than 50 minutes. After the food samples are cooked, the cooked food samples may be ground to a powder. For example, a pulverizer, grinder, or mortar may be used to grind the cooked food samples into a powder.

### EXTRACTING AND DIGESTING PROTEINS FROM FOOD SAMPLES AND DETERMINING THE PEANUT PEPTIDES IN FOOD SAMPLES

As discussed above, the concentration of non-peanut matrix proteins may be significantly greater than the concentration of peanut proteins in a food product. For example, only about 0.35 ppm Ara h 1 and about 1 ppm Ara h 3 are present in a 10 ppm peanut containing wheat flour food sample (i.e., as shown in Tables 3-5, below). This can mean that the amount of wheat flour proteins is 1,000,000 times greater than the amount of Ara h 1 and Ara h 3. If all wheat proteins are digested to peptides and analyzed using liquid chromatography with tandem mass spectrometry (LC-MSMS), competitive ionization will interfere with the identification and quantification of the peanut peptides in the food sample. This can lead to a suppression of the response of the peanut peptides determined by LC-MSMS, making it more difficult to identify and detect them from the peptide solution. Accordingly, methods for extracting proteins from the food sample, digesting proteins, and determining the relative response value of peanut peptides by LC-MSMS in food samples provided below are designed to suppress interferences that may be caused by non-peanut matrix proteins from the food sample.

The quantification of peanut proteins in each food sample is dependent upon the peptide markers that digest from the specific peanut proteins. Table 1, below, provides list of peptides specific to peanut proteins. Any or all of the peptides identified in Table 1 may be analyzed and/or quantified in a food sample.

TABLE 1
Peanut peptides

| Peptide | m/z (+1) | m/z (+2) | m/z (+3) | Protein |
|---|---|---|---|---|
| VLLEENAGGEQEER | 1572.75 | 786.8786 | 524.9215 | Ara h 1 |
| DLAFPGSGEQVEK | 1376.6692 | 688.8383 | 459.5613 | Ara h 1 |
| QQPEENACQFQR | 1477.6489 | 739.3281 | 493.2211 | Ara h 3 |
| CCNELNEFENNQR | 1612.6479 | 806.8276 | 538.2208 | Ara h 2 |
| NLPQQCGLR | 1028.5306 | 514.7689 | 343.515 | Ara h 2 |
| CDLDVSGGR | 921.4095 | 461.2084 | 307.808 | Ara h 6 |
| NLPQNCGFR | 1049.4993 | 524.7533 | 350.1713 | Ara h 7 |
| GTGNLELVAVR | 1128.6371 | 564.8222 | 376.8839 | Ara h 1 |
| VYDEELQEGHVLVVPQNFAVAGK | 2541.2984 | 1271.153 | 847.771 | Ara h 3 |
| CMCEALQQIMENQSDR | 1898.7864 | 949.8968 | 633.6003 | Ara h 2 |
| AHYQVVDSNGDR | 1360.624 | 680.8156 | 454.2129 | Ara h 3 |
| LNAQRPDNR | 1083.5654 | 542.2863 | 361.86 | Ara h 3 |
| IPSGFISYILNR | 1379.7682 | 690.3877 | 460.5942 | Ara h 1 |
| TANELNLLILR | 1269.7525 | 635.3799 | 423.9224 | Ara h 3 |
| RPFYSNAPQEIFIQQGR | 2051.0457 | 1026.027 | 684.3534 | Ara h 3 |
| NNPFYFPSR | 1141.5425 | 571.2749 | 381.1857 | Ara h 1 |
| SFNLDEGHALR | 1258.6175 | 629.8124 | 420.2107 | Ara h 1 |
| NTLEAAFNAEFNEIR | 1738.8395 | 869.9234 | 580.2847 | Ara h 1 |
| IFLAGDKDNVIDQIEK | 1817.9644 | 909.4858 | 606.6596 | Ara h 1 |
| TANDLNLLILR | 1255.7369 | 628.3721 | 419.2505 | Ara h 3 |
| AHVQVVDSNGDR | 1296.6291 | 648.8182 | 432.8812 | Ara h 3 |
| AQSENYEYLAFK | 1462.6849 | 731.8461 | 488.2331 | Ara h 3 |
| QFQNLQNHR | 1184.5919 | 592.7996 | 395.5355 | Ara h 1 |
| LFEVKPDDK | 1090.5779 | 545.7926 | 364.1975 | Ara h 1 |
| ANLRPCEQ | 930.4462 | 465.7267 | 310.8202 | Ara h 2 |
| NEFENNQR | 1050.4599 | 525.7336 | 350.8248 | Ara h 2 |
| QQWELQGDRR | 1315.6502 | 658.3287 | 439.2216 | Ara h 2 |
| ANLRPCEQHLMQK | 1567.7832 | 784.3952 | 523.2659 | Ara h 2 |

However, of the peptides listed in Table 1, not all are cooking-resistant or otherwise detectable for various reasons. Thus, some of the peptides of Table 1 may not be suitable for analysis in food products that have been cooked. Table 2, on the other hand, lists peanut peptides that are detectable and cooking resistant in their current condition. Any or all of the peptides listed in Table 2 may be suitable for analysis in food products that have been heated/cooked in their current condition.

TABLE 2
List of peanut peptides resistant to cooking/heating

| Peptide | Precursor | Quantifier | Qualifier | Protein |
|---|---|---|---|---|
| VLLEENAGGEQEER | 786.88 | 804.35 | 680.80 | Ara h 1 |
| DLAFPGSGEQVEK | 688.84 | 930.46 | 465.73 | Ara h 1 |
| GTGNLELVAVR | 564.82 | 686.4 | 557.5 | Ara h 1 |
| VYDEELQEGHVLVVPQNFAVAGK | 847.77 | 931.50 | 466.26 | Ara h 3 |
| TANELNLLILR | 635.38 | 741.50 | 854.58 | Ara h 3 |
| NNPFYFPSR | 571.27 | 821.38 | 1141.54 | Ara h 1 |
| SFNLDEGHALR | 629.81 | 797.4 | 682.4 | Ara h 1 |
| NTLEAAFNAEFNEIR | 869.92 | 1139.54 | 992.48 | Ara h 1 |
| IFLAGDKDNVIDQIEK | 606.66 | 779.41 | 722.87 | Ara h 1 |
| TANDLNLLILR | 628.37 | 1083.6 | 741.5 | Ara h 3 |
| AQSENYEYLAFK | 731.85 | 1263.59 | 722.84 | Ara h 3 |
| QFQNLQNHR | 592.80 | 583.79 | 554.28 | Ara h 1 |

Various preparation techniques may be utilized to prepare a food sample for analysis by quantification methods provided herein. Discussed below are preparation techniques including extraction, heating, digestion, purification, condensing, and reconstitution.

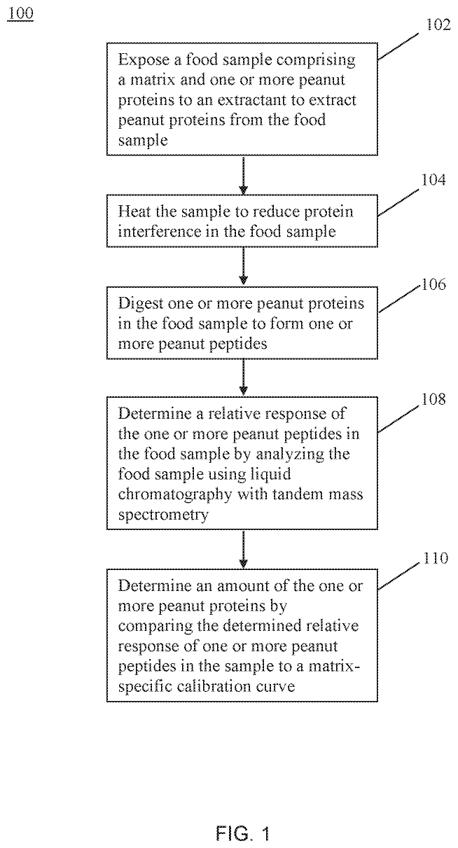

FIG. 1 provides a process diagram showing method 100 for quantifying peanut proteins in food products.

In step 102, a food sample comprising a matrix and one or more peanut proteins may be exposed to an extractant to extract proteins from the food sample. Suitable extractants (i.e., buffer solutions) may include a tris(hydroxymethyl)aminomethane buffer solution (“tris buffer”), or phosphate buffer solution (“PBS”). The buffer solution, or extractant, may be specific to the matrix. In some embodiments, exposing a food sample to an extractant can reduce the amount of the matrix in the food sample. In some embodiments, quantification methods provided herein may include more than one extraction step.

The pH of the extractant may improve the detectability of one or more peanut peptides when the sample is analyzed by LC-MSMS. In particular, optimizing the pH may maximize the extractability for peanut proteins. If the pH of the extractant is too high or too low, it may not effectively improve the sensitivity of the disclosed method and/or the detectability of one or more peanut peptides. In some embodiments, the pH of the extractant may be from 7 to 10 or from 7.5 to 8.5. In some embodiments, the pH of the extractant may be more than 7.0, more than 7.5, more than 8.0, more than 8.5, more than 9.0, or more than 9.5. In some embodiments, the pH of the extractant may be less than 10.0, less than 9.5, less than 9.0, less than 8.5, less than 8.0, or less than 7.5. In some embodiments, the pH of the extractant (i.e., buffer solution) may be specific to a particular matrix. In some embodiments, the pH of the extractant (i.e., buffer solution) may be specific to a matrix comprising wheat flour.

In step 104, the sample may be heated to reduce protein interference in the food sample. For example, heating the food sample may improve the detectability of one or more peanut peptides by reducing the “noise” introduced by non-peanut matrix proteins in the food sample. If the food samples are heated to a temperature that is too high, the heat may damage one or more peanut proteins and cause an inaccurate quantification result. If the samples are not heated enough, the interference of non-peanut matrix proteins may decrease the detectability of one or more peanut peptides. In some embodiments, the food sample may be heated to a temperature of 70-100° C. or from 70-85° C. In some embodiments, the food sample may be heated to a temperature of less than 100° C., less than 95° C., less than 90° C., less than 85° C., less than 80° C., or less than 75° C. In some embodiments, the food sample may be heated to a temperature of more than 70° C., more than 75° C., more than 80° C., more than 85° C., more than 90° C., or more than 95° C. In some embodiments, methods for quantification provided herein may include more than one heating steps.

In step 106, one or more peanut proteins in the food sample may be digested to form peanut peptides. The digestion of proteins can directly affect the measurement of one or more peanut peptides by mass spectrometry. In some embodiments, the amount of the digestive enzyme compared to the total amount of proteins may be optimized to improve the detectability of peanut peptides. In some embodiments, the digestive enzyme may be trypsin, chymotrypsin, or pepsin. Trypsin is a serine protease that hydrolyzes, or digests proteins. If the amount of the digestive enzyme in relation to the total amount of proteins in the food sample is too high or too low, it may not sufficiently digest the ideal number of proteins to optimize the detectability of the trace amounts of peanut peptides compared to the interference peptides (i.e., peptides digested from matrix proteins). In some embodiments, the amount of digestive enzyme may be 0.2-10 wt. % or 0.5-5 wt. % of the total amount of digestive enzyme and proteins. In some embodiments, the amount of digestive enzyme may be more than 0.2 wt. %, more than 0.5 wt. %, more than 0.8 wt. %, more than 1 wt. %, more than 1.5 wt. %, more than 2 wt. %, more than 2.5 wt. %, more than 3 wt. %, more than 3.5 wt. %, more than 4 wt. %, or more than 4.5 wt. % of the total amount of digestive enzyme and proteins. In some embodiments, 0.2-2 wt. % digestive enzyme of the total amount of digestive enzyme and proteins can improve the detectability of the peanut peptides. In some embodiments, fewer interference non-peanut proteins (i.e., matrix proteins) may be digested when a lesser amount of digestive enzyme is used to digest the proteins of a food sample (e.g., 0.2-2 wt. % trypsin of the total amount of digestive enzyme and proteins) which may result in improved detectability of peanut peptides.

In some embodiments, the amount of digestive enzyme used to digest proteins in a food sample may improve the sensitivity of disclosed methods of accurately detecting and quantifying peanut proteins in a food sample. For example, the mass spectrometry (i.e., LC-MSMS) response of a particular peptide may be calculated as the absolute peak area. As the absolute peak area increases, so too does the sensitivity for corresponding peanut protein.

After digestion, the sample may be purified. For example, the sample may be purified using solid phase extraction (SPE).

In some embodiments, nitrogen blowing or lyophilization may be used to condense a sample. For example, nitrogen may be introduced at 0.33 L/min for 2 hours from a nitrogen evaporator. In some embodiments, nitrogen blowing may be used to condense the sample by a factor of 2-20, 5-15, or 8-12. In some embodiments, nitrogen blowing may be used to condense the sample by a factor of ten. After nitrogen blowing, the sample may be reconstituted in solvent (e.g., water) and analyzed using LC-MSMS. The extent to which the sample is condensed may be specific to a particular food product.

Using nitrogen blowing to increase the sensitivity of detection method to the peanut peptides in a food sample can increase the detectability of peanut peptides when analyzed with LC-MSMS. For example, peanut peptides that may be more detectable using LC-MSMS include any of the peptides provided in Table 1 and/or Table 2. Specifically, the detectability of a particular peptide may be measured by the absolute peak area corresponding to that particular peptide.

After condensing the sample (e.g., with nitrogen blowing), the sample may be reconstituted with a reconstitution solvent.

In some embodiments, the composition of the reconstitution solvent may be optimized to improve the detectability of one or more peanut peptides and/or to achieve a more sensitive mass spectrometry response to a sample. For example, the composition of the reconstitution solvent may reduce the “noise” introduced by non-peanut matrix proteins/peptides and result in a more accurate quantification of the peanut proteins in a food product. In some embodiments, the reconstitution solvent may include acetonitrile, methanol, trifluoroacetic acid, formic acid, and/or water. If the amount of acetonitrile in the reconstitution solvent is too high or too low, the sensitivity of LC-MSMS to the peptide sample may not be optimized. In some embodiments, the reconstitution solvent may comprise 1-40% v/v, 5-30% v/v, or 10-20% v/v acetonitrile. In some embodiments, the reconstitution solvent may include more than 1% v/v, more than 5% v/v, more than 10% v/v, more than 15% v/v, more than 20% v/v, more than 25% v/v, more than 30% v/v, or more than 35% v/v acetonitrile. In some embodiments, the reconstitution solvent may include less than 40% v/v, less than 35% v/v, less than 30% v/v, less than 25% v/v, less than 20% v/v, less than 15% v/v, less than 10% v/v, or less than 5% v/v acetonitrile. The composition of the reconstitution solvent may be specific to a particular food product and/or matrix.

In step 108, a relative response value of the one or more peanut peptides in the food sample may be determined by analyzing the food sample using LC-MSMS. The relative peak areas corresponding to isotope-labeled peptides may be measured to quantify one or more peanut proteins in the food sample.

In step 110, an amount of the one or more peanut proteins may be determined by comparing the relative response value of one or more peanut peptides in the food sample determined by LC-MSMS to a matrix-specific calibration curve for the one or more peanut peptides. In some embodiments, the amount of peanut protein may be determined by determining the relative response value of a single peanut peptide by LC-MSMS. In some embodiments, the amount of peanut protein may be determined by determining the relative response values for two or more peanut peptides by LC-MSMS. As explained above, once the relative response value(s) for one or more peanut peptides has been determined by LC-MSMS, it may be evaluated with a calibration curve specific to the matrix being analyzed. Calibration curves according to embodiments provided herein are discussed in detail below.

### CALIBRATION CURVES FOR QUANTIFYING PEANUT PROTEINS IN FOOD PRODUCTS

In some embodiments, once a relative response value for one or more peptides in a food sample has been determined, as explained above, this determined relative response value can be compared to a calibration curve to determine an amount of peanut proteins in the food product. In some embodiments, a calibration curve may be specific to a particular matrix, or a particular matrix class (i.e., all matrices comprising the same primary ingredient). For example, a calibration curve may be specific to a matrix comprising wheat flour. In some embodiments, a calibration curve may include relative response values of peanut peptides over a range of peanut protein concentrations for the specific matrix.

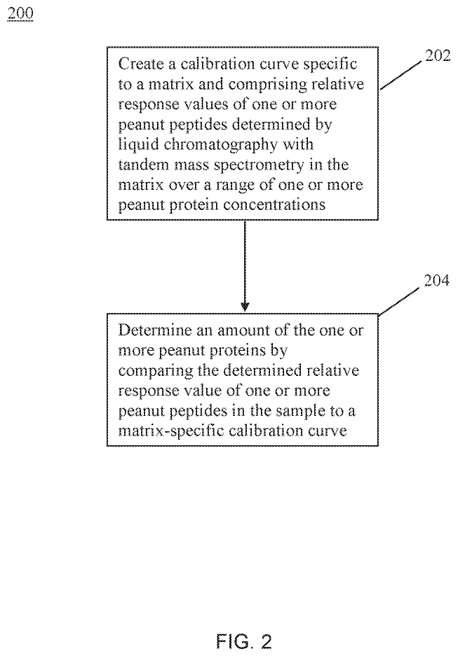

FIG. 2 shows a process diagram for a method 200 for quantifying an amount of peanut proteins in a food product. In step 202, a calibration curve may be created specifically for a matrix and comprise relative response values of one or more peanut peptides over a range of one or more peanut protein concentrations. For example, a calibration curve can be generated by plotting relative peak areas of peptide markers normalized with labeled peptide internal standards against concentrations of peanut proteins (e.g., Ara h 1, Ara h 3).

In some embodiments, a calibration curve may be developed using the relative response values of a specific peanut peptide (e.g., DLAFPGSGEQVEK) with a particular matrix along the y-axis and a range of concentrations for the particular peanut protein corresponding to the specific peanut peptide (e.g., Ara h 1) with the same matrix along the x-axis. Standard solutions of the peanut protein at various known concentrations are spiked into blank matrix extract. The spiked samples are digested, purified, and analyzed by LC-MSMS. The relative response of the particular peptide (e.g., DLAFPGSGEQVEK) is determined using the absolute peak area of the peptide divided by the absolute peak area of isotope labeled peptide (e.g., DLAFPGSGEQVEK (DLAFPGSGEQVE {Lys(13C6,15N2)})). Accordingly, once the relative response of one or more peptides (e.g., DLAFPGSGEQVEK) from a sample is determined, the concentration of Ara h 1 in the sample can be quantified using the calibration curve. By accounting for the matrix of a food product, a calibration curve can be generated specifically for a particular matrix. Because calibration curves provided herein may be specific to particular matrices, they can adjust the “noise” introduced into the food product by the non-peanut matrix ingredients and yield a more accurate quantification of peanut proteins in the given food product.

In step 204, an amount of the one or more peanut proteins may be determined by comparing the relative response value of one or more peanut peptides in the sample determined by LC-MSMS to a matrix-specific calibration curve.

By generating and applying a calibration curve that correlates the relative peak areas of peptide markers with peanut proteins, methods for quantifying peanut proteins in food samples provided herein eliminate a calculation required in current quantification methods (e.g., other LC-MSMS-based quantification methods). In particular, other LC-MSMS methods generally use calibration curves plotting the relative area of peptide markers against concentrations of peptide markers. The amount of peanut protein then needs to be calculated indirectly from the peptide concentrations, assuming that the molar concentration of the peanut peptides are equivalent to its corresponding peanut protein.

Accordingly, methods for quantifying peanut proteins in food products provided herein not only provide a more accurate method of quantifying peanut proteins in food products, but they also offer a more streamlined quantification process by eliminating a calculation step (i.e., indirectly calculating the peanut protein amounts from the peptide concentrations) required in other LC-MSMS quantification methods.

### EXAMPLES

Raw Food Product Compositions: Below, Tables 3-5 show various raw food samples that were prepared and used with methods for quantifying peanut proteins provided herein. Note that “RF” means raw wheat flour, “RFO” means raw wheat flour with hydrogenated soybean oil, and “RFOS” means raw wheat flour with hydrogenated soybean oil and salt.

TABLE 3
Example compositions of food samples comprising wheat flour.

| | Sample Name | | | | | | |
|---|---|---|---|---|---|---|---|
| | RF-80000 | RF-20000 | RF-4000 | RF-400 | RF-40 | RF-10 | RF-0 |
| Peanut powder | 8 g pure | 25 g RF- | 20 g RF- | 10 g RF- | 10 g RF- | 25 g RF- | 0 |
| | peanut | 80000 | 20000 | 4000 | 400 | 40 | |
| | powder | | | | | | |
| Wheat flour (g) | 92 | 75 | 80 | 90 | 90 | 75 | 100 |
| Peanut | 80,000 | 20,000 | 4,000 | 400 | 40 | 10 | 0 |
| Concentration | | | | | | | |
| (ppm) | | | | | | | |

TABLE 4
Example compositions of food samples comprising
wheat flour and hydrogenated soybean oil (HSO).

| | Sample Name | | | | | | |
|---|---|---|---|---|---|---|---|
| | RFO-80000 | RFO-20000 | RFO-4000 | RFO-400 | RFO-40 | RFO-10 | RFO-0 |
| Peanut powder | 8 g pure | 25 g RFO- | 20 g RFO- | 10 g RFO- | 10 g RFO- | 25 g RFO- | 0 |
| | peanut | 80000 | 20000 | 4000 | 400 | 40 | |
| | powder | | | | | | |
| Wheat flour (g) | 73.6 | 60 | 64 | 72 | 72 | 60 | 80 |
| HSO (g) | 18.4 | 15 | 16 | 18 | 18 | 15 | 20 |
| Peanut | 80,000 | 20,000 | 4,000 | 400 | 40 | 10 | 0 |
| Concentration | | | | | | | |
| (ppm) | | | | | | | |

TABLE 5
Example compositions of food samples comprising wheat
flour, hydrogenated soybean oil (HSO), and salt (NaCl).

| | Sample Name | | | | | | |
|---|---|---|---|---|---|---|---|
| | RFOS-80000 | RFOS-20000 | RFOS-4000 | RFOS-400 | RFOS-40 | RFOS-0 | RFOS-0 |
| Peanut powder | 8 g pure | 25 g RFOS- | 20 g RFOS- | 10 g RFOS- | 10 g RFOS- | 25 g RFOS- | 0 |
| | peanut | 80000 | 20000 | 4000 | 400 | 40 | |
| | powder | | | | | | |
| Wheat flour (g) | 72.68 | 59.25 | 63.2 | 71.1 | 71.1 | 59.25 | 79 |
| HSO (g) | 18.4 | 15 | 16 | 18 | 18 | 15 | 20 |
| NaCl (g) | 0.92 | 0.75 | 0.8 | 0.9 | 0.9 | 0.75 | 1 |
| Peanut | 80,000 | 20,000 | 4,000 | 400 | 40 | 10 | 0 |
| Concentration | | | | | | | |
| (ppm) | | | | | | | |

Peanut Protein Extraction, Digestion, and Peptide Screening Using LC-QTOF—Half a gram of a food sample was weighed and extracted using 5 mL tris buffer (pH 8.5) at 75° C. A Shaking Water Bath (Julabo) was used to extract the peanut proteins at 180 rpm for 2 hours. After extraction, the sample was centrifuged using Eppendorf 5810 centrifuge at 4000 rpm for 10 minutes and filtered with a Whatman filter paper. To quantify the total protein in the food sample, the filtrate was diluted 250× and analyzed using a Micro BCA Protein Assay Kit according to the manufacturer instructions. The original filtrate of 1 mL was digested with trypsin at a 1:40 enzyme to total protein concentration ratio at 37° C. overnight. Digestion was ceased by adding 511 μL 30% trichloroacetic acid. The solution was allowed to stand for 10 minutes, and then the solution was centrifuged at 14,000 rpm for 10 minutes.

The supernatant of the digestion solution was purified using solid phase extraction with an Agilent's Bond Elut Plexa column. Specifically, the Agilent's Bond Elut Plexa column was conditioned with 1 mL acetonitrile and equilibrated with 1 mL 1% formic acid (v/v). An amount of 1.4 mL digested sample solution was placed in the column. Interference compounds were washed away using 1 mL 1% formic acid (v/v) and the analyte was eluted with 1 mL 50% acetonitrile (v/v). After purification, the analyte was dried in nitrogen flow and reconstituted with 100 μL 5% acetonitrile (v/v).

High performance liquid chromatography (HPLC) was used to separate the particular peanut peptides in the food sample. Specifically, Agilent's HPLC 1290 system, equipped with a binary pump and Agilent's AdvanceBio Peptide Mapping column, was used to separate the peanut peptides in the food sample. The peanut peptides were eluted using two solvents—mobile phase A comprising 5% acetonitrile (v/v) with 0.05% formic acid, and mobile phase B comprising 95% acetonitrile (v/v) with 0.05% formic acid. The column flow rate was set at 0.3 mL/min with a column temperature of 50° C. The injection volume was 10 μL and the temperature of the autosampler was 10° C. Table 6 below shows the gradient elution condition of the AdvanceBio Peptide Mapping column.

TABLE 6
Gradient elution for peptide screening using LC-QToF

| Time (min) | A (%) | B (%) |
|---|---|---|
| 2 | 97 | 3 |
| 80 | 60 | 40 |
| 82 | 10 | 90 |
| 85 | 10 | 90 |
| 86 | 97 | 3 |
| 90 | 97 | 3 |

Auto MS/MS acquisition was performed on the digested food samples using an Agilent QTOF 6545 in positive mode. The temperature of the drying gas was set at 325° C., the flow rate of the drying gas was 9 L/min, the nebulizer was set at 45 psi, the temperature of the sheath gas was set at 275° C., the flow rate of the sheath gas was 11 L/min, and the capillary voltage was set at 4000 V. Further, the fragmentor, skimmer1, and octopoleRFPeak were set at 175 V, 65 V, and 750 V, respectively. The MS spectra were collected from 100 (m/z)-1700 (m/z) at a scan rate of 8 spectra/second. The MS/MS spectra were collected at a rate of 3 spectra/second, and the isolation of MS/MS was set at narrow. Ramped collision energy was used for auto MS/MS analysis. Ten precursor ions having a threshold over 3000 were identified during the tandem MS analysis. For precursor ions with two charges, slope and offset were 3.1 and 1, respectively. For precursor ions with three charges, slope and offset were 3.6 and −4.8, respectively. For precursor ions with more than three charges, slope and offset were 3.6 and −4.8, respectively.

Absolute peak area of peptides listed in Table 1 were measured and compared in raw and cooked samples. However, not all peptides yielded a consistent LC-MSMS response because some were not cooking resistant. Table 2 lists peanut peptides that are detectable and cooking resistant in current condition and further analyzed using LC-QQQ to select targeted peptides for method optimization and peanut quantification.

Targeted peptides selection: Using HPLC-QQQ, the peanut peptides of Table 2 were analyzed. Specifically, food samples comprising 10 ppm peanut and 0 ppm peanut were prepared to select targeted peptides for peanut quantification. Of the peptides provided in Table 2, DLAFPGSGEQVEK digested from Ara h 1, IFLAGDKDNVIDQIEK digested from Ara h 1, and TANDLNLLILR digested from Ara h 3 were each present in the 10 ppm peanut samples, each showed a constant mass spectrometry response in both raw and cooked food samples, and each showed a relatively high sensitivity compared to the other peanut peptides that were tested.

Once the above three peanut peptides (i.e., DLAFPGSGEQVEK digested from Ara h 1, IFLAGDKDNVIDQIEK digested from Ara h 1, and TANDLNLLILR digested from Ara h 3) were identified, multiple-reaction monitoring (MRM) parameters were optimized for use with quantification methods provided herein. (MRM is an acquisition mode of mass spectrometry.) Specifically, MRM parameters were optimized for synthesized peptides (specifically, synthesized peptides of the three peptides identified above) and isotope labelled peptides. Fragmentor and collision energy, were determined using MassHunter Optimizer for Peptides software (Agilent). Fragmentor range was set from 100 to 150 V with a step of 5. Cell accelerator voltage was set at 3 V. Positive ions with +H was selected as priority for precursor ions with charge state of 2 or 3. For product ion selection, four maximum number of product ions were included with low mass cut-off value of 80% precursor mass. The injection volume was 10 μL. After setting of peptide sequences, m/z of precursor ions and liquid chromatography method, MRM optimization started automatically using MassHunter Optimizer for Peptides software. The optimized MRM parameters are provided below, in Table 7.

TABLE 7
Optimized MRM parameters for peanut peptides used for quantification

| | | Precursor | Product | | CE | CAV | RT | Quantifier |
|---|---|---|---|---|---|---|---|---|
| Peptide sequence | Protein | (m/z) | (m/z) | Fragmentor | (V) | (V) | (min) | or Qualifier |
| DLAFPGSGEQVEK | Ara h 1 | 688.8 | 930.4 | 120 | 22 | 3 | 9.94 | Quantifier |
| | | | 465.7 | 120 | 25 | 3 | | Qualifier |
| DLAFPGSGEQVE{Lys(13C6, | | 692.8 | 938.5 | 120 | 21 | 3 | | Quantifier |
| 15N2)} | | | | | | | | |
| IFLAGDKDNVIDQIEK | Ara h 1 | 606.6 | 779.4 | 115 | 17 | 3 | 16.85 | Quantifier |
| | | | 722.9 | 120 | 15 | 3 | | Qualifier |
| IFLAGDKDNVIDQIE{Lys(13C6, | | 609.6 | 783.6 | 110 | 17 | 3 | | Quantifier |
| 15N2)} | | | | | | | | |
| TANDLNLLILR | Ara h3 | 628.4 | 741.5 | 125 | 21 | 3 | 21.58 | Quantifier |
| | | | 1083.6 | 125 | 21 | 3 | | Qualifier |
| TANDLNLLIL{Arg(13C6, | | 633.4 | 751.5 | 115 | 25 | 3 | | Quantifier |
| 15N4)} | | | | | | | | |

Peanut Protein Quantification Using LC-QQQ: One-half gram of a food sample was weighed and extracted using 5 mL tris buffer (pH 8.5) for 30 minutes using a Multi Reax shaker (heidolph). After heating at 75° C. for 30 minutes using a Shaker Water Bath (Julabo), the sample was centrifuged using Eppendorf 5810 centrifuge at 4000 rpm for 30 minutes and filtered with a Whatman filter paper. To quantify the total protein in the food sample, the filtrate was diluted 250× and analyzed using a Micro BCA Protein Assay Kit according to the manufacturer instructions. The original filtrate of 1 mL was digested with trypsin after adding 20 μL internal standard (heavy-labeled synthetic peptide, DLAFPGSGEQVE{Lys(13C6,15N2)} at 25 ng/mL, IFLAGDKDNVIDQIE{Lys(13C6,15N2)} at 25 ng/mL, and TANDLNLLIL{Arg(13C6,15N4)} at 62.5 ng/mL). The amount of trypsin used was 1:100 trypsin-to-total protein concentration ratio, and the sample was digested at 37° C. overnight. Digestion was ceased by adding 511 μL 30% trichloroacetic acid. The solution was allowed to stand for 10 minutes, and then the solution was centrifuged at 14,000 rpm for 10 minutes.

The supernatant of the digestion solution was purified using solid phase extraction with an Agilent's Bond Elut Plexa column. Specifically, the Agilent's Bond Elut Plexa column was conditioned with 1 mL acetonitrile and equilibrated with 1 mL 1% formic acid (v/v). An amount of 1.4 mL digested sample solution was placed in the column. Interference compounds were washed away using 1 mL 1% formic acid (v/v), and the analyte was eluted with 1 mL 50% acetonitrile (v/v). After purification, the analyte was dried in nitrogen flow and reconstituted with 100 μL 20% acetonitrile (v/v).

High performance liquid chromatography (HPLC) was used to separate the particular peanut peptides in the food sample. Specifically, Agilent's HPLC 1290 system, equipped with a binary pump and Agilent's AdvanceBio Peptide Mapping column, was used to separate the peanut peptides in the food sample. The peanut peptides were eluted using two solvents—mobile phase A comprising 5% acetonitrile (v/v) with 0.05% formic acid, and mobile phase B comprising 95% acetonitrile (v/v) with 0.05% formic acid. The column flow rate was set at 0.3 mL/min with a column temperature of 50° C. The injection volume was 10 μL and the temperature of the autosampler was 10° C. Table 8 below shows the gradient elution condition of the AdvanceBio Peptide Mapping column.

TABLE 8
Gradient elution for peptide detection using LC-QQQ

| Time (min) | A (%) | B (%) |
|---|---|---|
| 0 | 95 | 5 |
| 28 | 75 | 25 |
| 28.1 | 0 | 100 |
| 32 | 0 | 100 |
| 32.1 | 95 | 5 |
| 36 | 95 | 5 |

Tandem mass spectrometry (MS/MS) was performed on the digested food samples using multiple-reaction monitoring (MRM). Specifically, the method was performed using an Agilent QQQ 6470 in positive ionization mode. The temperature of the drying gas was 350° C., the flow rate of the drying gas was 9 L/min, the nebulizer was set at 45 psi, the temperature of the sheath gas was 380° C., the flow of the sheath gas was 11 L/min, the capillary voltage was 4000 V, and the nozzle voltage was 500 V.

The final LC-MSMS MRM method monitored three peptide transitions, including two transitions for each of the three native peptides and one transition for each of the three heavy-labeled synthetic peptides.

Generating Calibration Curve for Peanut Protein Quantification: A calibration curve showing the correlation between peanut peptides and their corresponding peanut proteins can be generated by plotting relative peak areas of peptide markers (normalized with labeled peptide internal standards) determined by LC-MSMS against concentrations of peanut proteins (e.g., Ara h 1, Ara h 3). The concentrations of peanut proteins (e.g., Ara h 1, Ara h 3) can be prepared by spiking in blank samples. Table 9, below, provides the concentrations of peanut proteins Ara h 1 and Ara h 3 in 1 mL blank sample extract that can be used to build a calibration curve.

TABLE 9
Concentration of Ara h 1 and Ara h
3 for building a calibration curve.

| | Concentration | | | | | | | | |
|---|---|---|---|---|---|---|---|---|---|
| | 9 | 8 | 7 | 6 | 5 | 4 | 3 | 2 | 1 |
| Ara h 1 | 0.55 | 1.09 | 2.19 | 4.38 | 8.75 | 17.5 | 35 | 70 | 140 |
| (ng/mL) | | | | | | | | | |
| Ara h 3 | 1.56 | 3.12 | 6.25 | 12.5 | 25 | 50 | 100 | 200 | 400 |
| (ng/mL) | | | | | | | | | |

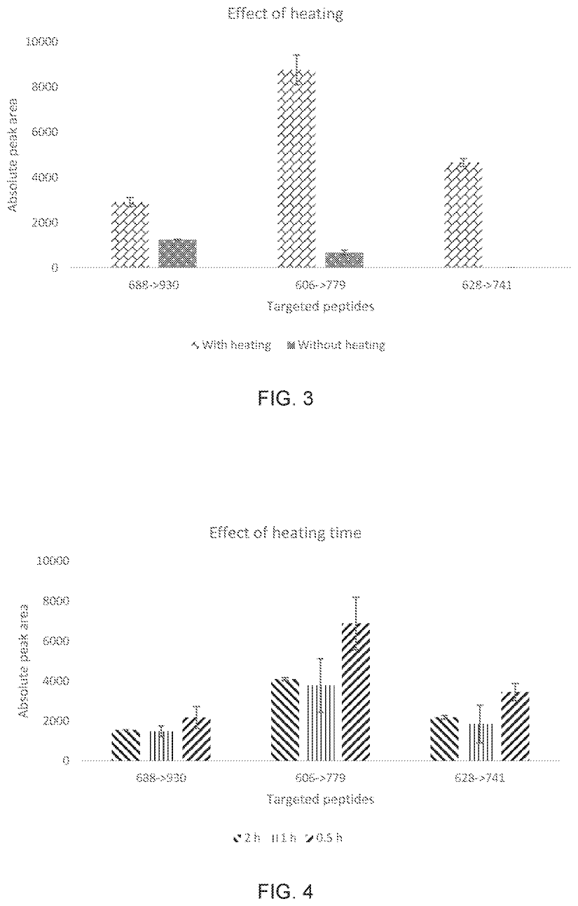

Heating Temperature: Because thermal stability of Ara h 1 and Ara h 3 has been reported up to 80° C., the effect of heating at 75° C. as a purification method was tested. The absolute mass spectrometry response of the targeted peptides (i.e., DLAFPGSGEQVEK, IFLAGDKDNVIDQIEK, and TANDLNLLILR) was used to illustrate the difference between sample preparation with or without heating. Three replicates were analyzed. The average mass spectrometry response was calculated and is provided in FIG. 3. As shown in FIG. 3, heating significantly increased the response of the targeted peanut peptides. For DLAFPGSGEQVEK and IFLAGDKDNVIDQIEK, (all digested from Ara h 1), the absolute peak area was 132% and 1187% greater, respectively, after heating. For TANDLNLLILR (digested from Ara h 3), the absolute peak area was 25,800% greater after heating.

Specifically, heating the food samples causes the interference proteins in the wheat flour to precipitate out of solution. However, because Ara h 1 and Ara h 3 proteins are stable up to 80° C., they are not affected by heating at 75° C.

Once it was determined that heating the food sample was a good way of increasing the detectability of peanut peptides, the time of heating was further optimized. In one trial, the results of which are reflected in FIG. 4. The length of time that the sample was heated varied. Specifically, the samples were heated for 0.5 hour, 1 hour, and 2 hours respectively.

As shown in FIG. 4, DLAFPGSGEQVEK and TANDLNLLILR all show an increased absolute peak when heated for 0.5 hours. The absolute peak decreases after this point. Accordingly, heating the samples for 0.5 hour results in the least amount of preparation time and highest amount of specificity.

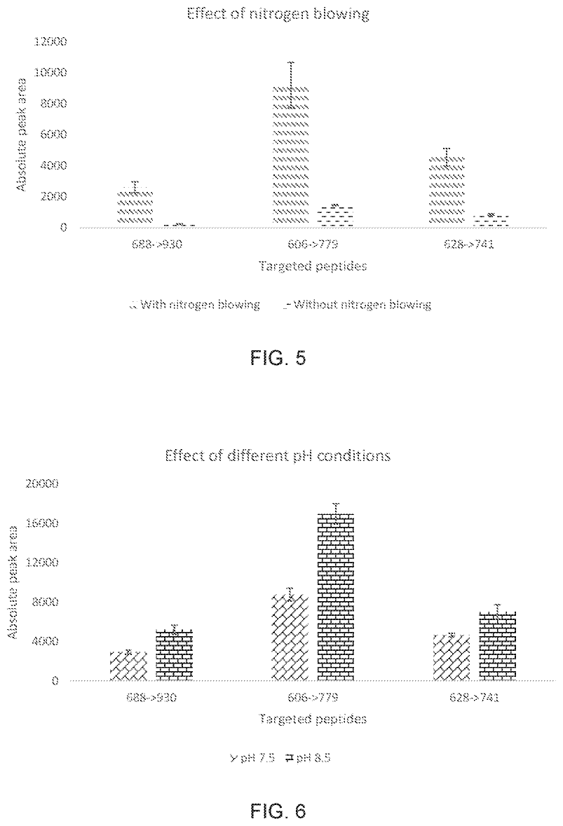

Nitrogen Blowing: FIG. 5 shows the mass spectrometry response results for peptide samples treated with nitrogen blowing and peptide samples not treated with nitrogen blowing.

To prepare the samples, a 1 mL peptide solution was dried with nitrogen blowing and reconstituted with 100 μL solvent. As shown in FIG. 5, the absolute areas of DLAFPGSGEQVEK and IFLAGDKDNVIDQIEK (digested from Ara h 1) were 1067% and 526% greater, respectively, after the solution was enriched using nitrogen blowing. The absolute area of TANDLNLLILR (digested from Ara h 3) was 419% greater after the solution was enriched with nitrogen blowing. Based on these results, nitrogen blowing may be used to improve the detectability of peanut peptides DLAFPGSGEQVEK, IFLAGDKDNVIDQIEK and TANDLNLLILR.

pH of Extractant: FIG. 6 provides the mass spectrometry response results when controlling the pH of the extraction solvent to improve the detectability of peanut proteins. A tris buffer having a pH of 7.5 was used to extract peanut proteins from a first sample of food sample, and a tris buffer having a pH of 8.5 was used to extract peanut proteins from a second sample of food product. The samples were testing using HPLC-MSMS. The detectability of a given peanut protein was measured as the absolute peak area. FIG. 6 provides the absolute peak areas for the peanut peptides DLAFPGSGEQVEK and IFLAGDKDNVIDQIEK (digested from Ara h 1) and TANDLNLLILR (digested from Ara h 3). As shown in FIG. 6, the absolute peak area for DLAFPGSGEQVEK was 78% higher using a tris buffer with a pH value of 8.5, the absolute peak area of IFLAGDKDNVIDQIEK was 94% higher using tris buffer with a pH of 8.5, and the absolute peak area of TANDLNLLILR was 50% higher using tris buffer having a pH of 8.5. These results suggest that a tris buffer having a pH of 8.5 may improve the detectability of peanut peptides DLAFPGSGEQVEK, IFLAGDKDNVIDQIEK and TANDLNLLILR more so than a tris buffer having a pH of 7.5.

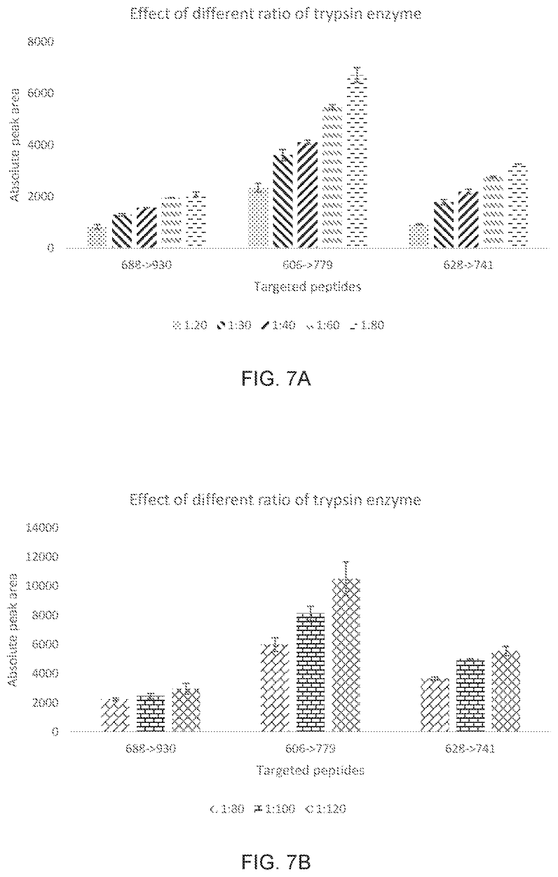

Trypsin Ratio: FIGS. 7A and 7B provide the mass spectrometry response results for peptide samples digested from various amounts of trypsin. Specifically, the trypsin-to-protein ratios that were tested include 1:20, 1:40, 1:60, 1:80, 1:100, and 1:120. As shown in FIG. 7B, the lowest amount of trypsin, 1:120 trypsin-to-protein ratio, resulted in the highest mass spectrometry response of the peanut peptides DLAFPGSGEQVEK, IFLAGDKDNVIDQIEK, and TANDLNLLILR. Although highest MS response of targeted peptides was achieved at 1:120, the standard deviation (SD) was also the highest compared with the other conditions. The ratio of 1:100 is chosen as the optimal trypsin to protein ratio, as a compromise of sensitivity and repeatability.

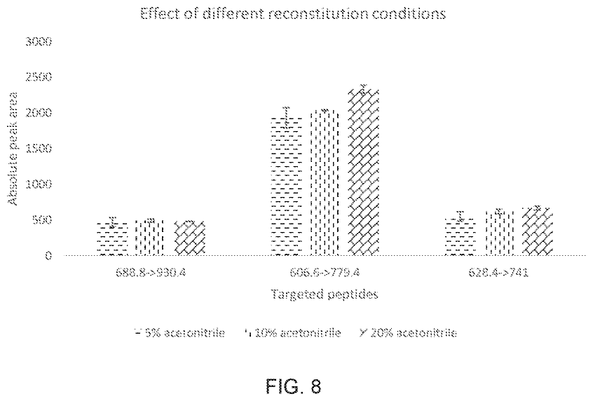

Reconstitution Solvent Composition: FIG. 8 provides the mass spectrometry response results for peptide samples reconstituted in solvents comprising various amounts of acetonitrile. In particular, peptide samples reconstituted in solvents comprising 5% v/v, 10% v/v, and 20% v/v were tested. As shown in FIG. 8, the peanut peptides IFLAGDKDNVIDQIEK and TANDLNLLILR showed an increase in detectability when 20% v/v acetonitrile was used. However, the detectability of the peanut peptide DLAFPGSGEQVEK was not much affected by the various amounts of acetonitrile in the reconstitution solvent.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

This application discloses several numerical ranges in the text and figures. The numerical ranges disclosed inherently support any range or value within the disclosed numerical ranges, including the endpoints, even though a precise range limitation is not stated verbatim in the specification because this disclosure can be practiced throughout the disclosed numerical ranges.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

1. A method of quantifying peanut proteins in a food sample comprising:
exposing a food sample comprising a matrix and one or more peanut proteins to an extractant to extract the one or more peanut proteins from the food sample;
heating the food sample to reduce protein interference in the food sample;
digesting one or more peanut proteins in the food sample to form one or more peanut peptides;
determining a relative response value of the one or more peanut peptides in the food sample by analyzing the food sample using liquid chromatography with tandem mass spectrometry (LC-MSMS); and
determining an amount of the one or more peanut proteins in the food sample by comparing the determined relative response value of the one or more peanut peptides in the food sample to a matrix-specific calibration curve.

2. The method of claim 1, wherein the matrix of the food sample comprises wheat flour.

3. The method of claim 1 or 2, wherein the matrix of the food sample comprises one or more interference proteins.

4. The method of claim 3, wherein exposing a food sample comprising a matrix and one or more peanut proteins to an extractant comprises extracting the one or more peanut proteins and one or more interference proteins from the food sample.

5. The method of any of claims 1-4, wherein the matrix-specific calibration curve is specific to a matrix comprising wheat flour.

6. The method of any of claims 1-5, wherein the matrix comprises one or more raw food ingredients.

7. The method of any of claims 1-6, wherein the matrix comprises one or more cooked food ingredients.

8. The method of any of claims 1-7, wherein the one or more peanut proteins comprises Ara h 1.

9. The method of any of claims 1-8, wherein the one or more peanut proteins comprises Ara h 3.

10. The method of any of claims 1-9, wherein heating the food sample to reduce protein interference in the food sample comprises heating the food sample to a temperature that is determined based on the matrix of the food sample.

11. The method of any of claims 1-10, wherein the matrix-specific calibration curve comprises LC-MSMS relative response values of the one or more peanut peptides in the matrix over a range of a peanut protein concentrations.

12. The method of any of claims 1-11, wherein the extractant comprises a tris buffer having a pH of 7.5-8.5.

13. A method of quantifying peanut proteins in a food sample comprising:
creating a calibration curve specific to a matrix and comprising liquid chromatography with tandem mass spectrometry (LC-MSMS) relative response values of one or more peanut peptides in the matrix over a range of one or more peanut protein concentrations; and
determining, from a food sample comprising a matrix and one or more peanut proteins, an amount of the one or more peanut proteins by comparing a relative response value of one or more peanut peptides in the food sample determined by LC-MSMS to the calibration curve.

14. The method of claim 13, wherein the matrix of the food sample comprises wheat flour.

15. The method of claim 13 or 14, wherein the matrix of the food sample comprises one or more interference proteins.

16. The method of any of claims 13-15, wherein the calibration curve comprises LC-MSMS relative response values of one or more peanut peptides in a matrix comprising wheat flour for a range of peanut protein concentrations.

17. The method of any of claims 13-16, wherein the matrix comprises one or more raw food ingredients.

18. The method of any of claims 13-17, wherein the matrix comprises one or more cooked food ingredients.

19. The method of any of claims 13-18, wherein the one or more peanut proteins comprises Ara h 1.

20. The method of any of claims 13-19, wherein the one or more peanut proteins comprises Ara h 3.

21. The method of any of claims 13-20, comprising heating the food sample to reduce protein interference in the food sample.

22. The method of claim 21, wherein heating the food sample comprises heating the food sample to a temperature that is determined based on the matrix of the food sample.

23. The method of any of claims 13-22, comprising exposing the food sample to an extractant to extract the one or more peanut proteins from the food sample.

24. The method of claim 23, wherein the extractant comprises a tris buffer having a pH of 7.5-8.5.

25. The method of any of claims 13-24, comprising digesting the one or more peanut proteins in the food sample to form one or more peanut peptides.

26. The method of any of claims 13-25, comprising determining a relative response value of the one or more peanut peptides in the food sample by analyzing the food sample using LC-MSMS.
